(19) United States
(12) Patent Application Publication
(10) Pub. No.: US 2021/0085190 A1
(43) Pub. Date: Mar. 25, 2021

(54) MOTION SENSOR-BASED PHYSIOLOGICAL PARAMETER OPTIMIZATION METHOD AND MONITORING DEVICE

(71) Applicants: SHENZHEN MINDRAY BIO-MEDICAL ELECTRONICS CO., LTD., Shenzhen (CN); SHENZHEN MINDRAY SCIENTIFIC CO., LTD., Shenzhen (CN)

(72) Inventors: Sanchao LIU, Shenzhen (CN); Qiang MA, Shenzhen (CN); Zehui SUN, Shenzhen (CN); Jian REN, Shenzhen (CN); Xianliang HE, Shenzhen (CN); Qiling LIU, Shenzhen (CN); Xingliang JIN, Shenzhen (CN); Zhigang YE, Shenzhen (CN); Hanyuan LUO, Shenzhen (CN); Ningling ZHANG, Shenzhen (CN)

(73) Assignees: SHENZHEN MINDRAY BIO-MEDICAL ELECTRONICS CO., LTD., Shenzhen (CN); SHENZHEN MINDRAY SCIENTIFIC CO., LTD., Shenzhen (CN)

(21) Appl. No.: 17/111,362

(22) Filed: Dec. 3, 2020

Related U.S. Application Data
(63) Continuation of application No. PCT/CN2018/089960, filed on Jun. 5, 2018.

(51) Int. Cl. A61B 5/024; A61B 5/11; A61B 5/00

(52) U.S. Cl. CPC A61B 5/024; A61B 5/1118; A61B 2562/0219; A61B 5/7225; A61B 5/0002; A61B 5/7221

(57) ABSTRACT

A method and a monitoring device for optimizing physiological parameter using a motion sensor are provided. The method includes synchronously acquiring a physiological signal and a motion signal of a patient; determining a signal characteristic of the physiological signal from the physiological signal, and determining whether the physiological signal is reliable according to the signal characteristic of the physiological signal; when determining the physiological signal is unreliable, determining a signal characteristic of the motion signal from the motion signal, and determining a motion state during each heartbeat period of a monitored object according to the signal characteristic of the motion signal; according to the motion state during the heartbeat period, determining an effectiveness of the physiological signal during the heartbeat period, or correcting the signal characteristic of the physiological signal during the heartbeat period when calculating physiological parameters, or directly correcting physiological parameters.

### CROSS REFERENCE TO RELATED APPLICATIONS

This disclosure is a bypass continuation of Patent Cooperation Treaty Application No. PCT/CN2018/089960, filed on Jun. 5, 2018, which is incorporated herein by reference in its entirety.

### TECHNICAL FIELD

The disclosure relates to the field of medical apparatuses, and in particular to a method for optimizing physiological parameter using a motion sensor and a monitoring device.

### BACKGROUND

With the development of medical technologies and improvement of people's cognition of medicine, the importance and attention of rapid recovery after operation have been sharply enhanced and promoted. During the postoperative recovery period, medical personnel hope that patients can get out of bed and do more activities, to promote rapid physical recovery. A conventional bedside monitoring device limits an activity space of the patient, and the patient cannot do activities comfortably due to lengthy and complicated cables. When the patients do activities, a physiological signal of the patient may also be monitored, but the patient's activities may interfere with the monitored physiological signal, so that the physiological signal of the patient doing activities cannot be accurately monitored.

Therefore, the current monitoring device still has many limitations.

### SUMMARY

In one aspect, a method for optimizing physiological parameter using a motion sensor is provided. The method includes acquiring a physiological signal of a monitored object by a physiological sensor attached to the monitored object, and synchronously acquiring a motion signal of the monitored object by a motion sensor attached to the monitored object; determining a signal characteristic of the physiological signal from the physiological signal, and determining whether the physiological signal is reliable according to the signal characteristic of the physiological signal; when determining the physiological signal is unreliable, determining a signal characteristic of the motion signal from the motion signal, and determining a motion state during each heartbeat period of the monitored object during a time period in which the motion signal is acquired according to the signal characteristic of the motion signal; and according to the motion state during the heartbeat period, determining an effectiveness of the physiological signal during the heartbeat period, or correcting the signal characteristic of the physiological signal during the heartbeat period when calculating a physiological parameter of the monitored object, or directly correcting the physiological parameter of the monitored object.

In an embodiment, providing a signal characteristic extraction unit that is configured to extract and output a signal characteristic of an input signal inputted to the signal characteristic extraction unit; where the signal characteristic extraction unit is configured to extract and output the signal characteristic of the physiological signal when the input signal is the physiological signal, and extract and output the signal characteristic of the motion signal when the input signal is the motion signal.

In an embodiment, before the signal characteristic of the physiological signal is determined from the physiological signal and the signal characteristic of the motion signal is determined from the motion signal, the method further includes: preprocessing the physiological signal and the motion signal.

In an embodiment, preprocessing the physiological signal and the motion signal includes: performing at least one of filtering, amplification, and analog-to-digital conversion on the physiological signal and the motion signal.

In an embodiment, determining whether the physiological signal is reliable according to the signal characteristic of the physiological signal includes: assigning a weight to each signal characteristic of the physiological signal, acquiring a reliability score of the physiological signal through voting and scoring, and determining whether the physiological signal is reliable based on the reliability score.

In an embodiment, determining a motion state during each heartbeat period of the monitored object during a time period in which the motion signal is acquired according to the signal characteristic of the motion signal includes: grading the motion state during each heartbeat period of the monitored object during the time period in which the motion signal is acquired according to the signal characteristic of the motion signal, to obtain a motion grade during each heartbeat period of the monitored object.

In an embodiment, grading the motion state during each heartbeat period of the monitored object during the time period in which the motion signal is acquired according to the signal characteristic of the motion signal, to obtain a motion grade during each heartbeat period of the monitored object includes: calculating a mean value or a variance of the signal characteristics of the motion signal, and grading the motion state during each heartbeat period of the monitored object during a time period in which the motion signal is acquired through threshold segmentation, to obtain the motion grade during each heartbeat period of the monitored object.

In an embodiment, the motion grade includes: a low motion grade for indicating that the monitored object has no motion or slight motion, a high motion grade for indicating that the monitored object has intense motion, and a medium motion grade between the low motion grade and the high motion grade.

In an embodiment, determining an effectiveness of the physiological signal during the heartbeat period, or correcting the signal characteristic of the physiological signal during the heartbeat period when calculating a physiological parameter of the monitored object, or directly correcting the physiological parameter of the monitored object, according to the motion state during the heartbeat period includes: determining an effectiveness of the physiological signal according to the motion grade during the heartbeat period, or correcting the signal characteristic of the physiological signal during the heartbeat period according to the motion grade during the heartbeat period when calculating the physiological parameter of the monitored object, or directly correcting the physiological parameter according to the motion grade during the heartbeat period of the monitored object.

In an embodiment, the signal characteristic includes signal quality; and correcting the signal characteristic of the physiological signal during the heartbeat period according to the motion grade during the heartbeat period when calculating the physiological parameter of the monitored object includes: correcting the signal quality of the physiological signal during the heartbeat period according to the motion grade during the heartbeat period when calculating the physiological parameter of the monitored object.

In an embodiment, determining an effectiveness of the physiological signal according to the motion grade during the heartbeat period includes: when the motion grade during the heartbeat period is a low motion grade for indicating that the monitored object has no motion or slight motion, determining that the physiological signal during the heartbeat period is effective; when the motion grade during the heartbeat period is a high motion grade for indicating that the monitored object has intense motion, determining that the physiological signal during the heartbeat period is ineffective; and when the motion grade during the heartbeat period is a medium motion grade between the low motion grade and the high motion grade, reconfirming the effectiveness of the physiological signal during the heartbeat period.

In an embodiment, correcting the signal characteristic of the physiological signal during the heartbeat period according to the motion grade during the heartbeat period when calculating the physiological parameter of the monitored object includes: when the motion grade during the heartbeat period is a low motion grade for indicating that the monitored object has no motion or slight motion, the signal characteristic of the physiological signal is not modified when calculating the physiological parameter of the monitored object; and when the motion grade during the heartbeat period is a high motion grade for indicating that the monitored object has intense motion or a medium motion grade between the low motion grade and the high motion grade, correcting the signal characteristic of the physiological signal when calculating the physiological parameter of the monitored object.

In an embodiment, the signal characteristic includes a time domain characteristic and/or a frequency domain characteristic of a signal; and the time domain characteristic of the signal includes: at least one of a peak position, a peak amplitude, a peak slope, a peak width, peak effectiveness, a peak type, a peak-to-peak interval value, peak-to-peak interval effectiveness, and signal quality.

In an embodiment, correcting the signal characteristic of the physiological signal during the heartbeat period when calculating a physiological parameter of the monitored object includes: correcting at least one weight of a time domain characteristic or a frequency domain characteristic of the physiological signal during the heartbeat period when calculating the physiological parameter of the monitored object.

In an embodiment, when determining the physiological signal is unreliable, the method further includes: directly calculating the physiological parameter of the monitored object based on the signal characteristic of the physiological signal determined from the physiological signal; and determining whether the calculated physiological parameter during the heartbeat period is effective according to the effectiveness of the physiological signal during the heartbeat period.

In an embodiment, the method further includes: when determining the physiological signal is reliable, directly calculating the physiological parameter based on the signal characteristic of the physiological signal.

In another aspect, a monitoring device is provided. The medical device includes a physiological sensor configured to be attached to a monitored object to acquire a physiological signal of the monitored object; a motion sensor configured to be attached to the monitored object to acquire a motion signal of the monitored object; and a processor configured to: determine a signal characteristic of the physiological signal from the physiological signal, and determine , whether the physiological signal is reliable according to the signal characteristic of the physiological signal; if the physiological signal is unreliable, determine a signal characteristic of the motion signal from the motion signal, and determine a motion state during each heartbeat period of the monitored object during a time period in which the motion signal is acquired according to the signal characteristic of the motion signal; and according to the motion state during the heartbeat period, determine an effectiveness of the physiological signal during the heartbeat period, or correct the signal characteristic of the physiological signal during the heartbeat period when calculating a physiological parameter of the monitored object, or directly correct the physiological parameter of the monitored object.

In an embodiment, the processor includes a signal characteristic extraction unit configured to: extract an input signal and output a signal characteristic of the input signal, extract a physiological signal and output a signal characteristic of the physiological signal when the input signal is the physiological signal, and extract a motion signal and output a signal characteristic of the motion signal when the input signal is the motion signal.

In an embodiment, the monitoring device further includes a preprocessing circuit configured to first preprocess the physiological signal and the motion signal before the processor determines the signal characteristic of the physiological signal from the physiological signal and determines the signal characteristic of the motion signal from the motion signal.

In an embodiment, the preprocessing circuit includes at least one of the following: a filter circuit configured to filter an input signal; an amplification circuit configured to amplify an input signal; and an analog-to-digital conversion circuit configured to perform analog-to-digital conversion on an input signal.

In an embodiment, determining, by the processor according to the signal characteristic of the physiological signal, whether the physiological signal is reliable includes: assigning a weight to each signal characteristic of the physiological signal, acquiring a reliability score of the physiological signal through voting and scoring, and determining whether the physiological signal is reliable based on the reliability score.

In an embodiment, determining, by the processor, a motion state during each heartbeat period of the monitored object during a time period in which the motion signal is acquired according to the signal characteristic of the motion signal includes: grading the motion state during each heartbeat period of the monitored object during the time period in which the motion signal is acquired according to the signal characteristic of the motion signal, to obtain a motion grade during each heartbeat period of the monitored object.

In an embodiment, grading, by the processor, the motion state during each heartbeat period of the monitored object during a time period in which the motion signal is acquired according to the signal characteristic of the motion signal, to obtain a motion grade during each heartbeat period of the monitored object includes: calculating a mean value or a variance of the signal characteristics of the motion signal, and grading the motion state during each heartbeat period of the monitored object during a time period in which the motion signal is acquired through threshold segmentation, to obtain the motion grade during each heartbeat period of the monitored object.

In an embodiment, the motion grade includes: a low motion grade for indicating that the monitored object has no motion or slight motion, a high motion grade for indicating that the monitored object has intense motion, and a medium motion grade between the low motion grade and the high motion grade.

In an embodiment, determining, by the processor, determining an effectiveness of the physiological signal during the heartbeat period, or correcting the signal characteristic of the physiological signal during the heartbeat period when calculating a physiological parameter of the monitored object, or directly correcting the physiological parameter of the monitored object, according to the motion state during the heartbeat period includes: determining an effectiveness of the physiological signal according to the motion grade during the heartbeat period, or correcting the signal characteristic of the physiological signal during the heartbeat period according to the motion grade during the heartbeat period when calculating the physiological parameter of the monitored object, or directly correcting the physiological parameter according to the motion grade during the heartbeat period of the monitored object.

In an embodiment, the signal characteristic includes signal quality; and correcting the signal characteristic of the physiological signal during the heartbeat period according to the motion grade during the heartbeat period when calculating the physiological parameter of the monitored object includes: correcting the signal quality of the physiological signal during the heartbeat period according to the motion grade during the heartbeat period when calculating the physiological parameter of the monitored object.

In an embodiment, determining, by the processor, effectiveness of the physiological signal according to the motion grade during the heartbeat period includes: when the motion grade during the heartbeat period is a low motion grade for indicating that the monitored object has no motion or slight motion, determining that the physiological signal during the heartbeat period is effective; when the motion grade during the heartbeat period is a high motion grade for indicating that the monitored object has intense motion, determining that the physiological signal during the heartbeat period is ineffective; and when the motion grade during the heartbeat period is a medium motion grade between the low motion grade and the high motion grade, reconfirming the effectiveness of the physiological signal during the heartbeat period.

In an embodiment, correcting the signal characteristic of the physiological signal during the heartbeat period according to the motion grade during the heartbeat period when calculating the physiological parameter of the monitored object includes: when the motion grade during the heartbeat period is a low motion grade for indicating that the monitored object has no motion or slight motion, the signal characteristic of the physiological signal is not modified when calculating the physiological parameter of the monitored object; and when the motion grade during the heartbeat period is a high motion grade for indicating that the monitored object has intense motion or a medium motion grade between the low motion grade and the high motion grade, correcting the signal characteristic of the physiological signal when calculating the physiological parameter of the monitored object.

In an embodiment, the signal characteristic includes a time domain characteristic and/or a frequency domain characteristic of a signal; and the time domain characteristic of the signal includes: at least one of a peak position, a peak amplitude, a peak slope, a peak width, peak effectiveness, a peak type, a peak-to-peak interval value, peak-to-peak interval effectiveness, and signal quality.

In an embodiment, correcting, by the processor, correcting the signal characteristic of the physiological signal during the heartbeat period when calculating a physiological parameter of the monitored object includes: correcting at least one weight of a time domain characteristic or a frequency domain characteristic of the physiological signal during the heartbeat period when calculating the physiological parameter of the monitored object.

In an embodiment, when determining the physiological signal is unreliable, the method further includes: directly calculating the physiological parameter of the monitored object based on the signal characteristic of the physiological signal determined from the physiological signal; and determining whether the calculated physiological parameter during the heartbeat period is effective according to the effectiveness of the physiological signal during the heartbeat period.

In an embodiment, when determining the physiological signal is reliable, directly calculating the physiological parameter based on the signal characteristic of the physiological signal.

In an embodiment, the monitoring device further includes a housing, where the processor is arranged in the housing and is connected to the physiological sensor and the motion sensor in a wired or wireless manner.

In an embodiment, the motion sensor includes at least one of an acceleration sensor, an angular velocity sensor, or a gravity sensor.

In an embodiment, the monitoring device is a mobile monitoring device.

In another aspect, a computer-readable storage medium is provided. The computer-readable storage medium includes a program that can be executed by a processor to implement the above described methods for optimizing physiological parameter using a motion sensor.

In the medical device of this disclosure, a physiological signal and a motion signal of a patient are synchronously acquired, a signal characteristic of the physiological signal is determined from the physiological signal, and whether the physiological signal is reliable is determined according to the signal characteristic of the physiological signal; when determining the physiological signal is unreliable, a signal characteristic of the motion signal is determined from the motion signal, and determining a motion state during each heartbeat period of the monitored object during a time period in which the motion signal is acquired according to the signal characteristic of the motion signal; and an effectiveness of the physiological signal during the heartbeat period is determined according to the motion state during the heartbeat period, or the signal characteristic of the physiological signal during the heartbeat period is corrected when a physiological parameter of the patient is calculated, thereby improving accuracy of the physiological signal and the physiological parameter.

### BRIEF DESCRIPTION OF THE DRAWINGS

Following detailed descriptions of respective embodiments in this disclosure can be understood better when combining with these figures, in which the same structure is represented by the same reference sign. In the figures:

FIG. 1 is a system framework diagram of a parameter processing module in a multi-parameter monitor;

FIG. 2 is a system framework diagram of a parameter processing module in a single-parameter monitor;

FIG. 3 is a schematic structural diagram of a monitor networking system used in a hospital;

FIG. 4 is a flowchart of a method for optimizing physiological parameter using a motion sensor according to an embodiment;

FIG. 5 is a flowchart of a method for optimizing physiological parameter using a motion sensor according to another embodiment;

FIG. 6 is a schematic diagram of correcting a signal characteristic of a physiological signal during a heartbeat period according to a motion state during the heartbeat period when calculating a physiological parameter according to an embodiment;

FIG. 7 is a schematic structural diagram of a monitoring device according to an embodiment;

FIG. 8 is a schematic structural diagram of a monitoring device according to another embodiment;

FIG. 9 is a schematic structural diagram of a monitoring device according to still another embodiment; and

FIG. 10 is a schematic structural diagram of a monitoring device according to yet another embodiment.

### DETAILED DESCRIPTION

The disclosure will be further described in detail below through specific implementations in conjunction with the accompanying drawings. Associated similar element reference numerals are used for similar elements in different implementations. In the following implementations, many details are described such that the present application can be better understood. However, it would have been effortlessly appreciated by a person skilled in the art that some of the features could be omitted or could be substituted by other elements, materials, and methods in different cases. In certain cases, some operations involved in the present application are not displayed or described in the specification, which is to prevent a core part of the present application from being obscured by too much description. Moreover, for a person skilled in the art, the detailed description of the involved operations is not necessary, and the involved operations can be thoroughly understood according to the description in the specification and the general technical knowledge in the art.

In addition, the characteristics, operations, or features described in the specification can be combined in any appropriate manner to form various implementations. Moreover, the steps or actions in the method description can also be exchanged or adjusted in order in a way that would have been obvious to a person skilled in the art. Therefore, the various orders in the specification and the accompanying drawings are merely for the purpose of clear description of a certain embodiment and are not meant to be a necessary order unless otherwise stated that a certain order must be followed.

The serial numbers themselves for the components herein, for example, “first” and “second”, are merely used to distinguish the described objects, and do not have any sequential or technical meaning. Moreover, as used in the present application, “connection” or “coupling”, unless otherwise specified, includes both direct and indirect connections (couplings).

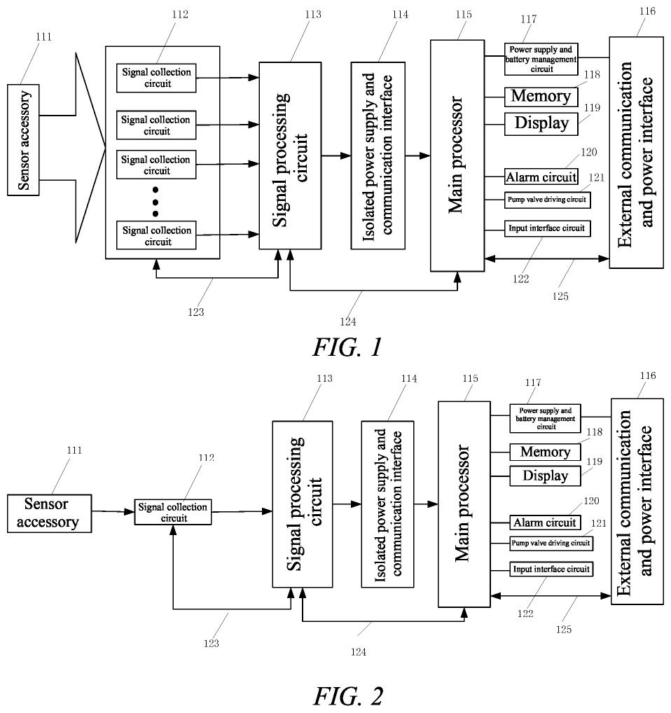

As shown in FIG. 1, a system framework diagram of a parameter processing module in a multi-parameter monitor is provided. The multi-parameter monitor has an independent housing, and a sensor interface area is arranged on a housing panel. A plurality of sensor interfaces are integrated in the sensor interface area and configured to be connected to various external physiological parameter sensor accessories 111. The housing panel further includes a small IXD display area, a display 119, an input interface circuit 122, an alarm circuit 120 (such as an LED alarm area), and the like. The parameter processing module has an external communication and power interface for communicating with a host and obtaining power from the host. The parameter processing module further supports an externally inserted parameter module. The parameter module may be inserted to form a plug-in monitor host as a part of the monitor, or may be connected to the host via a cable, where the externally inserted parameter module serves as an external accessory of the monitor.

An internal circuit of the parameter processing module is placed in the housing. As shown in FIG. 1, the internal circuit includes signal collection circuits 112 corresponding to at least two physiological parameters, a front-end signal processing circuit 113, and a main processor 115. The signal collection circuit 112 may be selected from an electrocardiogram circuit, a respiration circuit, a body temperature circuit, a blood oxygen circuit, a non-invasive blood pressure circuit, an invasive blood pressure circuit, and the like. These signal collection circuits 112 are respectively electrically connected to corresponding sensor interfaces, so as to be electrically connected to sensor accessories 111 corresponding to different physiological parameters. An output end of the signal collection circuit is coupled to a front-end signal processor, a communication port of the front-end signal processor is coupled to the main processor, and the main processor is electrically connected to the external communication and power interface. Various physiological parameter measurement circuits can use common circuits in the prior art. The front-end signal processing circuit completes sampling and analog-to-digital conversion of an output signal of the signal collection circuit, and outputs a control signal to control a measurement process of a physiological signal. These parameters include but are not limited to: parameters such as electrocardiogram, respiration, body temperature, blood oxygen, non-invasive blood pressure, and invasive blood pressure. The front-end signal processing circuit may be implemented by using a single-chip microcomputer or other semiconductor devices, for example, by using a single-chip microcomputer, an ASIC, or an FPGA. The front-end signal processing circuit may also be powered by an isolated power supply, and data obtained through sampling may be sent to the main processor through an isolated communication interface after being simply processed and packaged. For example, the front-end signal processing circuit 113 may be coupled to the main processor 115 through an isolated power supply and communication interface 114. The reason why the front-end signal processing circuit is powered by the isolated power supply is that the DC/DC power supply isolated by a transformer plays a role in isolating a patient from a power supply apparatus, with the main purposes of: 1. isolating the patient, and enabling an application part to be floating by means of an isolation transformer, so that a leakage current of the patient is small enough; and 2. preventing the voltage or energy during defibrillation or electrotome application from affecting a board card and a device of an intermediate circuit such as a main control board (guaranteed by a creepage distance and electrical clearance). Certainly, the front-end signal processor circuit 113 may also be connected to the main processor 115 by means of a cable 124. The main processor completes calculation of the physiological parameter, and sends a calculation result and waveforms of the parameter to the host (such as a host with a display, a PC, and a central station) through the external communication and power interface 116. The main processor 115 may also be connected to the external communication and power interface 116 by means of a cable 125, and the external communication and power interface 116 may be one of local area network interfaces composed of the Ethernet, a token ring, a token bus, and a fiber distributed data interface (FDDI) as the backbone network of these three networks, or a combination thereof, or may be one of wireless interfaces such as an infrared interface, a Bluetooth interface, a Wi-Fi interface, and a WMTS communication interface, or a combination thereof, or may be one of wired data connection interfaces such as an RS232 interface and a USB interface, or a combination thereof. The external communication and power interface 116 may also be one of a wireless data transmission interface and a wired data transmission interface or a combination thereof. The host may be any computer apparatus such as a host of a monitor, an electrocardiograph, an ultrasonic diagnostic apparatus, and a computer, and after being installed with matching software, the host can form a monitoring apparatus. The host may further be a communication apparatus such as a mobile phone, and the parameter processing module sends, by using a Bluetooth interface, data to the mobile phone supporting Bluetooth communication, so as to implement remote transmission of the data. After completing the calculation of the physiological parameter, the main processor 115 may further determine whether the physiological parameter is abnormal, and if yes, may give an alarm by means of the alarm circuit 120. In addition, a power supply and battery management circuit 117 in the figure is configured to manage and process power supply of the monitor, and a memory 118 may store intermediate and final data of the monitor, and store a program instruction or code executed by the main processor 115 and the like. If the monitor has a function of blood pressure measurement, the monitor may further include a pump valve driving circuit 121. The pump valve driving circuit 121 is configured to perform inflation or deflation operations under the control of the main processor 115. If the monitor does not have the function of blood pressure measurement, the pump valve driving circuit 121 may be eliminated.

As shown in FIG. 2, a processing system architecture of a monitor with a single physiological parameter is provided. For the same content, reference may be made to the above content.

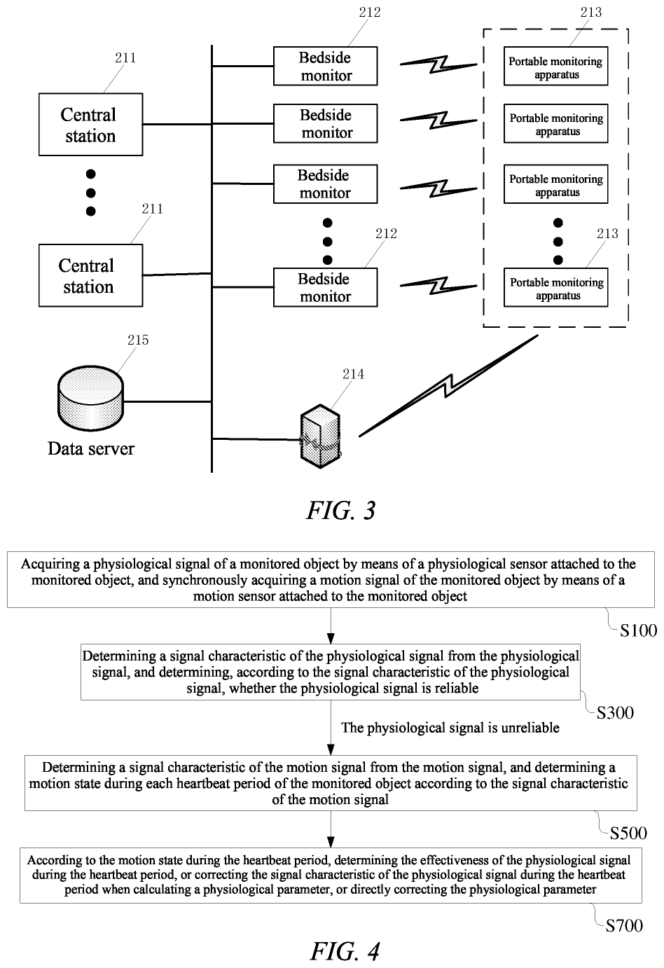

As shown in FIG. 3, a monitor networking system used in a hospital is provided. By using the system, data of the monitor may be saved as a whole to centrally manage patient information and nursing information that are stored in association, which facilitates storage of historical data and alarming in association. In the system shown in FIG. 3, a bedside monitor 212 may be provided for each hospital bed. The bedside monitor 212 may be the above multi-parameter monitor or a plug-in monitor. In addition, each bedside monitor 212 may further be paired with one portable monitoring apparatus 213 for transmission. The portable monitoring apparatus 213 provides a simple and portable parameter processing module, and the simple and portable parameter processing module may be worn on the body of a patient to perform mobile monitoring for the patient. After the portable monitoring apparatus 213 and the bedside monitor 212 perform wired or wireless communication, physiological data generated through mobile monitoring may be transmitted to the bedside monitor 212 for display, or transmitted, by using the bedside monitor 212, to a central station 211 for a doctor or a nurse to check, or transmitted to a data server 215 for storage by using the bedside monitor 212. In addition, the portable monitoring apparatus 213 may further directly transmit, by using a wireless network node 214 arranged in the hospital, the physiological data generated through mobile monitoring to the central station 211 for storage and display, or transmit, by using the wireless network node 214 arranged in the hospital, the physiological data generated through mobile monitoring to the data server 215 for storage. It can be seen that the data corresponding to the physiological parameter displayed on the bedside monitor 212 may originate from a sensor accessory directly connected to the monitor, or from the portable monitoring apparatus 213, or from the data server. The portable monitoring apparatus 213 may be connected to the sensor accessory 111 in a wired and/or wireless manner, and include a part or all of the circuits of the above parameter processing module. For example, isolation measures for isolating patients may not be provided in the portable monitoring apparatus 213 but provided outside the portable monitoring apparatus 213, for example, provided on the sensor accessory 111. The portable monitoring apparatus 213 may be equipped with a display screen for displaying a parameter calculation result and/or alarm prompt information. For example, the portable monitoring apparatus 213 may be a wireless sensor patch attached to the body, or a transfer monitor, or a telemetry apparatus.

Referring to FIG. 4, an embodiment provides a method for optimizing physiological parameter using a motion sensor (hereinafter referred to as a physiological parameter optimization method). The method may be applied to the monitor mentioned above, and includes steps S100 to S700, which will be described in detail below.

Step S100, acquiring a physiological signal of a monitored object by a physiological sensor attached to the monitored object, and synchronously acquiring a motion signal of the monitored object by a motion sensor attached to the monitored object.

In an embodiment, the motion sensor is a sensor configured to sense the motion of the monitored object. For example, the motion sensor may include at least one of a speed sensor, an acceleration sensor, an angular velocity sensor (such as a gyroscope), or a gravity sensor, and corresponding motion signals are respectively a speed signal (which may be a velocity vector with a direction or a rate without a direction), an acceleration signal, an angular velocity signal, and a gravitational acceleration signal. Certainly, the motion signal may also be a duration of the motion. The motion sensor is attached to the monitored object, and may be directly attached to the body or clothes of the monitored object. For example, the motion sensor may be placed on an electrode lead wire, or placed on a device adjacent to the electrode/lead wire, or integrated with the electrode. When the monitor is a mobile monitor, the motion sensor may be arranged on a circuit board in the monitor.

Step S300, determining a signal characteristic of the physiological signal from the physiological signal, and determining whether the physiological signal is reliable according to the signal characteristic of the physiological signal.

In an embodiment, determining, according to the signal characteristic of the physiological signal, whether the physiological signal is reliable may include: assigning a weight to each signal characteristic of the physiological signal, acquiring a reliability score of the physiological signal through voting and scoring, and determining whether the physiological signal is reliable based on the reliability score. For example, by means of a machine learning algorithm, a weight is assigned to each signal characteristic of the physiological signal, the reliability score of the physiological signal is acquired by voting and scoring on these signal characteristics of the physiological signal, and then whether the physiological signal is reliable is determined based on the reliability score. For example, a reliability threshold is set. If the reliability score is greater than the reliability threshold, it is determined that the physiological signal is reliable; otherwise, it is determined that the physiological signal is unreliable. For example, it is assumed that the physiological signal has three signal characteristics, that is, signal quality, matching of QRS complexes, and effectiveness of QRS complexes, and weights may be respectively set for the three signal characteristics. For example, a weight of the signal quality is 50%, a weight of the matching of QRS complexes is 30%, and a weight of the effectiveness of QRS complexes is 20%. Then voting and scoring are performed on the three signal characteristics. For example, higher signal quality leads to a higher score of the signal quality. For example, the full score is 10 points, and higher signal quality leads to a score of the signal quality being closer to 10 points. Similarly, better matching of QRS complexes leads to a higher score of the matching of QRS complexes, and better effectiveness of QRS complexes leads to a higher score of the effectiveness of QRS complexes. Certainly, simple division may be performed for the matching of QRS complexes and the effectiveness of QRS complexes, where if the matching of QRS complexes is good, a score is obtained, and if the matching of QRS complexes is not good, another score is obtained, or if a QRS complex is effective, a score is obtained, and if the QRS complex is ineffective, another score is obtained. Then, scores of the three signal characteristics are multiplied by the weights thereof and products are added to obtain the reliability score of the physiological signal, and finally whether the physiological signal is reliable is determined based on the reliability score. Certainly, a plurality of thresholds about reliability may also be set, and then reliability grade classification is performed on the physiological signal according to the reliability score of the physiological signal, and finally the physiological signal is normalized to be reliable or unreliable.

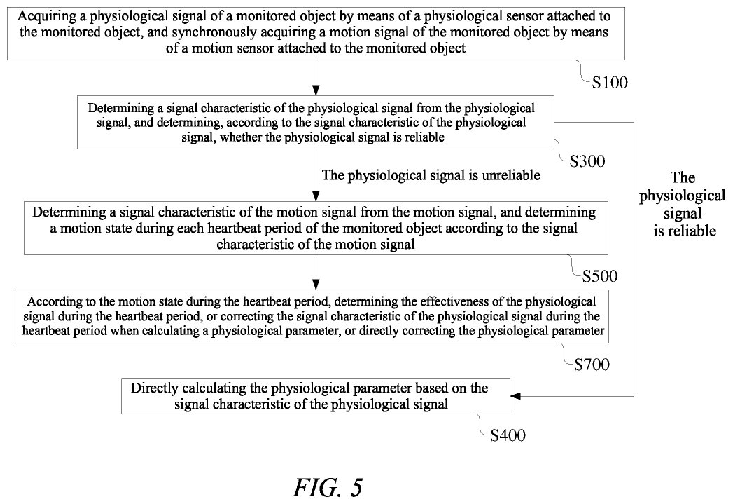

Referring to FIG. 5, in an embodiment, the physiological parameter optimization method of the disclosure may further include step S400, when determining that the physiological signal is reliable, directly calculating a physiological parameter based on the signal characteristic of the physiological signal.

Step S500, when determining the physiological signal is unreliable, determining a signal characteristic of the motion signal from the motion signal, and determining a motion state during each heartbeat period of the monitored object during a time period in which the motion signal is acquired according to the signal characteristic of the motion signal. The heartbeat period represents a duration of a heartbeat, for example, the heartbeat period may be understood as a QRS complex interval.

In an embodiment, determining a motion state during each heartbeat period of the monitored object according to the signal characteristic of the motion signal may include: grading the motion state during each heartbeat period of the monitored object during a time period in which the motion signal is acquired according to the signal characteristic of the motion signal, to obtain a motion grade during each heartbeat period of the monitored object. For example, a mean value and/or a variance of signal characteristics of the motion signal may be calculated, and the motion state during each heartbeat period of the monitored object may be graded through threshold segmentation, to obtain the motion grade during each heartbeat period of the monitored object. For example, motion signals are graded by setting different segmentation thresholds.

In an embodiment, the motion grade may include three grades, for example, a low motion grade for indicating that the monitored object has no motion or slight motion, a high motion grade for indicating that the monitored object has intense motion, and a medium motion grade between the low motion grade and the high motion grade. In other embodiments, the motion grade may also be divided into other numbers of grades, for example, two grades (for example, only the low motion grade and the high motion grade are retained) and four grades.

Step S300 involves the signal characteristic of the physiological signal, and step S500 involves the signal characteristic of the motion signal. In an embodiment, the signal characteristic may include a time domain characteristic and/or a frequency domain characteristic of a signal. The time domain characteristic of the signal may include: at least one of a peak position, a peak amplitude, a peak slope, a peak width, peak effectiveness, a peak type, a peak-to-peak interval value, peak-to-peak interval effectiveness, and signal quality. There are many specific implementations. For example, the time domain signal is used as an example, where peak searching may be performed on the signal, a peak amplitude, a peak slope, and a peak width may be calculated, and signal quality and other time domain characteristics such as peak effectiveness, a peak type, a peak-to-peak interval value, and interval effectiveness may be further calculated. For example, the frequency domain characteristic is used as an example, where Fourier Transform may be performed on the signal to obtain a frequency characteristic of the signal, and then a high-frequency signal and a low-frequency signal are processed to obtain a required spectrum characteristic. In some embodiments, in order to better extract the signal characteristic of the signal, the signal may further be filtered and denoised first, for example, power frequency interference, baseline drift, and high frequency noise interference of the signal are filtered out.

It can be seen that, according to the method of the disclosure, the signal characteristic of the physiological signal and the signal characteristic of the motion signal are introduced in a physiological parameter optimization process, and the purpose of physiological parameter optimization is finally achieved with the signal characteristics of the two types of signals. However, in a specific implementation process, a signal characteristic extraction unit may be provided and configured to: extract and output a signal characteristic of an input signal inputted to the signal characteristic extraction unit, extract a physiological signal and output a signal characteristic of the physiological signal when the input signal is the physiological signal, and extract a motion signal and output a signal characteristic of the motion signal when the input signal is the motion signal. The signal characteristic of the physiological signal and the signal characteristic of the motion signal are extracted by using a same hardware structure, so that hardware unlocking can be effectively reduced, and costs can be reduced.

In an embodiment, in order to preferably obtain the signal characteristic of the signal, before the signal characteristic of the physiological signal is determined from the physiological signal and the signal characteristic of the motion signal is determined from the motion signal, preprocessing the physiological signal and the motion signal, for example the physiological signal and the motion signal are first preprocessed in parallel, for example, at least one of filtering, amplification, and analog-to-digital conversion may be performed on the signal. For example, the physiological sensor and the motion sensor respectively acquire the physiological signal and the motion signal. The two types of signals are first filtered to remove noise and then amplified to enlarge signal amplitudes. Finally, the physiological signal and the motion signal are converted from analog signals into digital signals through analog-to-digital conversion.

Step S700, according to the motion state during the heartbeat period, determining an effectiveness of the physiological signal during the heartbeat period, or correcting the signal characteristic of the physiological signal during the heartbeat period when calculating a physiological parameter of the monitored object, or directly correcting the physiological parameter of the monitored object.

In an embodiment, determining an effectiveness of the physiological signal during the heartbeat period according to the motion state during the heartbeat period may include: determining an effectiveness of the physiological signal according to the motion grade during the heartbeat period. For example, when the motion grade during the heartbeat period is the low motion grade, it may be determined that the physiological signal during the heartbeat period is effective; when the motion grade during the heartbeat period is the medium motion grade, the effectiveness of the physiological signal during the heartbeat period is reconfirmed; and when the motion grade during the heartbeat period is the high motion grade, it is determined that the physiological signal during the heartbeat period is ineffective. The reconfirmation of the effectiveness of the physiological signal during the heartbeat period may be further confirming the effectiveness of these physiological signals in combination with the motion characteristic of the motion signal, or reconfirming the effectiveness of these physiological signals after the physiological signals are corrected.

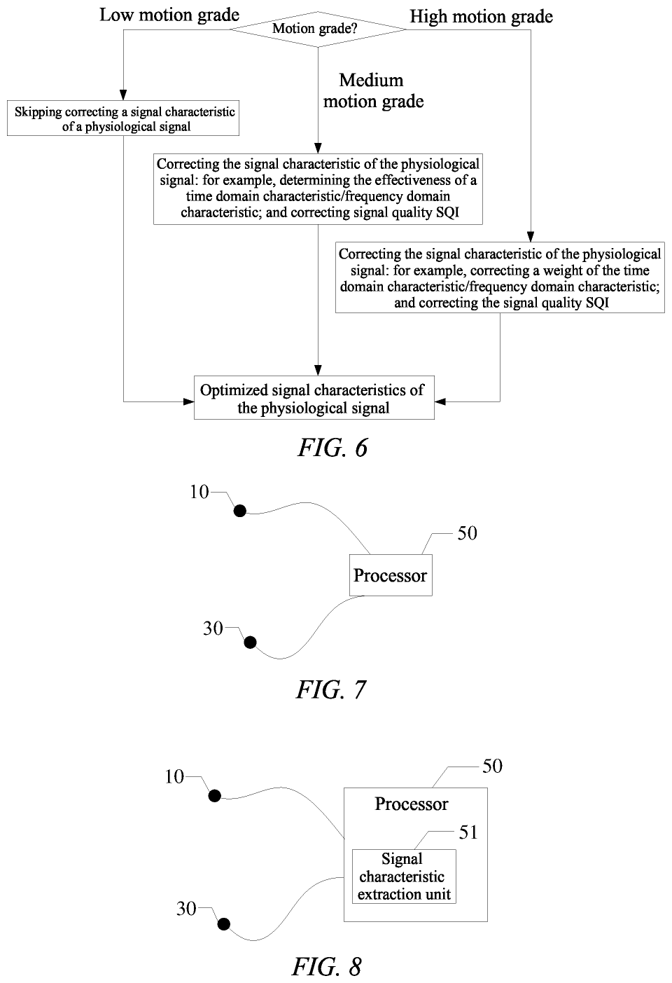

Referring to FIG. 6, in an embodiment, correcting the signal characteristic of the physiological signal during the heartbeat period according to the motion state during the heartbeat period when calculating a physiological parameter of the monitored object may include: correcting the signal characteristic of the physiological signal during the heartbeat period according to the motion grade during the heartbeat period when calculating the physiological parameter of the monitored object. For example, when the motion grade during the heartbeat period is the low motion grade, the signal characteristic of the physiological signal is not corrected; and when the motion grade during the heartbeat period is the medium motion grade or the high motion grade, the signal characteristic of the physiological signal is corrected. For example, when the motion grade during the heartbeat period is the medium motion grade, effectiveness of a time domain characteristic and/or a frequency domain characteristic of the physiological signal is determined, and a grade/threshold of the signal quality may also be corrected; and when the motion grade during the heartbeat period is the high motion grade, weights/a weight (which may be set to 0) of the time domain characteristic and/or the frequency domain characteristic of the physiological signal are/is corrected, and the grade/threshold of the signal quality may also be corrected. Alternatively, for example, signal quality of the physiological signal during the heartbeat period may be corrected according to the motion grade during the heartbeat period when calculating the physiological parameter. The signal characteristic may include the signal quality, for example, a grade or threshold of the signal quality may be corrected. For example, when the signal characteristic of the physiological signal is determined from the physiological signal, signal quality is obtained, and then a value of the signal quality may be corrected according to the motion grade during the heartbeat period. For another example, a corresponding value may be pre-stored for each motion grade, and when the signal characteristic of the physiological signal is determined from the physiological signal, signal quality is obtained, and the obtained signal quality may be directly replaced according to a value corresponding to the motion grade during the heartbeat period. Alternatively, for example, weights/a weight of a time domain characteristic and/or a frequency domain characteristic of the physiological signal during the heartbeat period may be corrected when the physiological parameter is calculated. The signal characteristic may include a time domain characteristic and/or a frequency domain characteristic of a signal.

However, in another embodiment, when the motion grade during the heartbeat period is the low motion grade, it may be determined that the physiological signal during the heartbeat period is effective (therefore, the physiological parameter of the monitored object generated according to the physiological signal is also effective). When the motion grade during the heartbeat period is the medium motion grade, the physiological signal (such as a QRS complex interval, an value, and a QRS complex type) is processed, for example, correction processing is performed. When the motion grade during the heartbeat period is the high motion grade, the physiological parameter (a heart rate value, arrhythmia, a blood oxygen value, and blood oxygen saturation) generated according to the physiological signal is directly processed, for example, correction processing is performed. In this embodiment, it may be directly determined, according to the motion grade, that the physiological signal is effective and therefore the physiological parameter is also effective, the physiological signal may be corrected, or the physiological parameter may be corrected directly.

In an embodiment, the physiological parameter optimization method of the disclosure may further include a step. If it is determined that the physiological signal is unreliable, the method further includes: directly calculating the physiological parameter based on the signal characteristic of the physiological signal determined from the physiological signal; and determining whether the calculated physiological parameter during the heartbeat period is effective according to the effectiveness of the physiological signal during the heartbeat period. For example, after the physiological parameter is directly calculated based on the physiological signal characteristic determined from the physiological signal, if the motion grade is the low motion grade, it is determined that the directly calculated physiological parameter is effective, if the motion grade is the medium motion grade, it is determined that the directly calculated physiological parameter is in doubt, and if the motion grade is the high motion grade, it is determined that the directly calculated physiological parameter is ineffective.

The above are some content of the physiological parameter optimization method of the disclosure. According to the method, a physiological signal of a monitored object is acquired, and a signal characteristic of the physiological signal is determined based on the physiological signal of the monitored object, and then reliability of the physiological signal is determined based on the signal characteristic of the physiological signal. In addition, a motion signal of the monitored object is acquired, and a signal characteristic of the motion signal is determined based on the motion signal of the monitored object, so as to further determine a motion state during each heartbeat period of the monitored object. When it is determined that the physiological signal is unreliable, optimization is performed according to the motion state during the heartbeat period, thereby improving accuracy of a physiological parameter and reducing false alarms.

Referring to FIG. 7, an embodiment of the disclosure further discloses a monitoring device or a monitor, and the monitoring device or the monitor may include a physiological sensor 10, a motion sensor 30, and a processor 50. In an embodiment, the monitoring device further includes a housing, where the processor 50 is arranged in the housing and is connected to the physiological sensor 10 and the motion sensor 30 in a wired or wireless manner. Detailed description is given below.

The physiological sensor 10 is configured to be attached to a monitored object to acquire a physiological signal of the monitored object.

The motion sensor 30 is configured to be attached to the monitored object to acquire a motion signal of the monitored object. In an embodiment, the motion sensor is a sensor configured to sense the motion of the monitored object. For example, the motion sensor may include at least one of a speed sensor, an acceleration sensor, an angular velocity sensor (such as a gyroscope), or a gravity sensor, and corresponding motion signals are respectively a speed signal (which may be a velocity vector with a direction or a rate without a direction), an acceleration signal, an angular velocity signal, and a gravitational acceleration signal. Certainly, the motion signal may also be a duration of the motion. The motion sensor is attached to the monitored object, and may be directly attached to the body or clothes of the monitored object, or may be attached to the housing of the monitoring device. For example, the motion sensor may be placed on an electrode lead wire, or placed on a device adjacent to the electrode/lead wire, or integrated with the electrode. When the monitor is a mobile monitor, the motion sensor may be arranged on a circuit board in the monitor.

The processor 50 is configured to: determine a signal characteristic of the physiological signal from the physiological signal, and determine whether the physiological signal is reliable according to the signal characteristic of the physiological signal; when the physiological signal is unreliable, determine a signal characteristic of the motion signal from the motion signal, and determine a motion state during each heartbeat period of the monitored object during a time period in which the motion signal is acquired according to the signal characteristic of the motion signal; and according to the motion state during the heartbeat period, determine an effectiveness of the physiological signal during the heartbeat period, or correct the signal characteristic of the physiological signal during the heartbeat period when calculating a physiological parameter of the monitored object, or directly correct the physiological parameter of the monitored object. In an embodiment, the processor 50 may acquire the physiological signal of the monitored object by the physiological sensor attached to the monitored object, and synchronously acquire the motion signal of the monitored object by the motion sensor attached to the monitored object.

From the description above, the processor 50 first determines reliability of the physiological signal; and performs optimization with the motion signal when determining that the physiological signal is unreliable, which is to be described in detail below.

When determining the reliability of the physiological signal, the processor 50 may first determine the signal characteristic of the physiological signal from the physiological signal, and determine whether the physiological signal is reliable according to the signal characteristic of the physiological signal. For example, the processor 50 may assign a weight to each signal characteristic of the physiological signal, acquiring a reliability score of the physiological signal through voting and scoring, and determine whether the physiological signal is reliable based on the reliability score. Specifically, for example, by means of a machine learning algorithm, a weight is assigned to each signal characteristic of the physiological signal, the reliability score of the physiological signal is acquired by voting and scoring on these signal characteristics of the physiological signal, and then whether the physiological signal is reliable is determined based on the reliability score. For example, a reliability threshold is set. If the reliability score is greater than the reliability threshold, it is determined that the physiological signal is reliable; otherwise, it is determined that the physiological signal is unreliable. For example, it is assumed that the physiological signal has three signal characteristics, that is, signal quality, matching of QRS complexes, and effectiveness of QRS complexes, and weights may be respectively set for the three signal characteristics. For example, a weight of the signal quality is 50%, a weight of the matching of QRS complexes is 30%, and a weight of the effectiveness of QRS complexes is 20%. Then voting and scoring are performed on the three signal characteristics. For example, higher signal quality leads to a higher score of the signal quality. For example, the full score is 10 points, and higher signal quality leads to a score of the signal quality being closer to 10 points. Similarly, better matching of QRS complexes leads to a higher score of the matching of QRS complexes, and better effectiveness of QRS complexes leads to a higher score of the effectiveness of QRS complexes. Certainly, simple division may be performed for the matching of QRS complexes and the effectiveness of QRS complexes, where if the matching of QRS complexes is good, a score is obtained, and if the matching of QRS complexes is not good, another score is obtained, or if a QRS complex is effective, a score is obtained, and if the QRS complex is ineffective, another score is obtained. Then, scores of the three signal characteristics are multiplied by the weights thereof and products are added to obtain the reliability score of the physiological signal, and finally whether the physiological signal is reliable is determined based on the reliability score. Certainly, a plurality of thresholds about reliability may also be set, and then reliability grade classification is performed on the physiological signal according to the reliability score of the physiological signal, and finally the physiological signal is normalized to be reliable or unreliable.

When determining the physiological signal is reliable, the processor 50 directly calculates the physiological parameter based on the signal characteristic of the physiological signal; and when determining the physiological signal is unreliable, the processor 50 performs subsequent optimization.

When determining the physiological signal is unreliable, the processor 50 determines the signal characteristic of the motion signal from the motion signal, and determines the motion state during each heartbeat period of the monitored object during a time period in which the motion signal is acquired according to the signal characteristic of the motion signal. For example, in an embodiment, the processor 50 may grade the motion state during each heartbeat period of the monitored object according to the signal characteristic of the motion signal, to obtain a motion grade during each heartbeat period of the monitored object. For example, the processor 50 may calculate a mean value and/or a variance of signal characteristics of the motion signal, and grade the motion state during each heartbeat period of the monitored object through threshold segmentation, to obtain the motion grade during each heartbeat period of the monitored object during a time period in which the motion signal is acquired. For example, the processor 50 grades motion signals by setting different segmentation thresholds. In an embodiment, the motion grade may include three grades, for example, a low motion grade for indicating that the monitored object has no motion or slight motion, a high motion grade for indicating that the monitored object has intense motion, and a medium motion grade between the low motion grade and the high motion grade. In other embodiments, the motion grade may also be divided into other numbers of grades, for example, two grades (for example, only the low motion grade and the high motion grade are retained) and four grades.

According to the motion state during the heartbeat period, the processor 50 then determines the effectiveness of the physiological signal during the heartbeat period, or corrects the signal characteristic of the physiological signal during the heartbeat period when calculating the physiological parameter of the monitored object, or directly corrects the physiological parameter.

In an embodiment, determining, by the processor 50, an effectiveness of the physiological signal during the heartbeat period according to the motion state during the heartbeat period may include: determining an effectiveness of the physiological signal according to the motion grade during the heartbeat period. For example, when the motion grade during the heartbeat period is the low motion grade, the processor 50 may determine that the physiological signal during the heartbeat period is effective; when the motion grade during the heartbeat period is the medium motion grade, the processor reconfirms the effectiveness of the physiological signal during the heartbeat period; and when the motion grade during the heartbeat period is the high motion grade, the processor determines that the physiological signal during the heartbeat period is ineffective. The reconfirmation of the effectiveness of the physiological signal during the heartbeat period may be further confirming the effectiveness of these physiological signals in combination with the motion characteristic of the motion signal, or reconfirming the effectiveness of these physiological signals after the physiological signals are corrected.

In an embodiment, correcting, by the processor 50, the signal characteristic of the physiological signal according to the motion state during the heartbeat period during the heartbeat period when calculating a physiological parameter of the monitored object may include: correcting the signal characteristic of the physiological signal during the heartbeat period according to the motion grade during the heartbeat period when calculating the physiological parameter of the monitored object. For example, when the motion grade during the heartbeat period is the low motion grade, the processor 50 does not correct the signal characteristic of the physiological signal; and when the motion grade during the heartbeat period is the medium motion grade or the high motion grade, the processor corrects the signal characteristic of the physiological signal. For example, when the motion grade during the heartbeat period is the medium motion grade, effectiveness of a time domain characteristic and/or a frequency domain characteristic of the physiological signal is determined, and a grade/threshold of signal quality may also be corrected; and when the motion grade during the heartbeat period is the high motion grade, weights/a weight (which may be set to 0) of the time domain characteristic and/or the frequency domain characteristic of the physiological signal are/is corrected, and the grade/threshold of the signal quality may also be corrected. Alternatively, for example, the processor 50 may correct signal quality of the physiological signal during the heartbeat period according to the motion grade during the heartbeat period when calculating the physiological parameter. The signal characteristic may include the signal quality, for example, a grade or threshold of the signal quality may be corrected. For example, when the signal characteristic of the physiological signal is determined from the physiological signal, signal quality is obtained, and then a value of the signal quality may be corrected according to the motion grade during the heartbeat period. For another example, a corresponding value may be pre-stored for each motion grade, and when the signal characteristic of the physiological signal is determined from the physiological signal, signal quality is obtained, and the obtained signal quality may be directly replaced according to a value corresponding to the motion grade during the heartbeat period. Alternatively, for example, the processor 50 may correct weights/a weight of a time domain characteristic and/or a frequency domain characteristic of the physiological signal during the heartbeat period when calculating the physiological parameter. The signal characteristic may include a time domain characteristic and/or a frequency domain characteristic of a signal.

However, in another embodiment, when the motion grade during the heartbeat period is the low motion grade, the processor 50 may determine that the physiological signal during the heartbeat period is effective (therefore, the physiological parameter generated according to the physiological signal is also effective). When the motion grade during the heartbeat period is the medium motion grade, the physiological signal (such as a QRS complex interval, an value, and a QRS complex type) is processed, for example, correction processing is performed. When the motion grade during the heartbeat period is the high motion grade, the physiological parameter (a heart rate value, arrhythmia, a blood oxygen value, and blood oxygen saturation) generated according to the physiological signal is directly processed, for example, correction processing is performed. In this embodiment, it may be directly determined, according to the motion grade, that the physiological signal is effective and therefore the physiological parameter is also effective, the physiological signal may be corrected, or the physiological parameter may be corrected directly.

In an embodiment, when determining the physiological signal is unreliable, the processor 50 may further directly calculating the physiological parameter of the monitored object based on the signal characteristic of the physiological signal determined from the physiological signal; and determining whether the calculated physiological parameter during the heartbeat period is effective according to the effectiveness of the physiological signal during the heartbeat period. For example, after directly calculating the physiological parameter based on the physiological signal characteristic determined from the physiological signal, if the motion grade is the low motion grade, the processor 50 determines that the directly calculated physiological parameter is effective, if the motion grade is the medium motion grade, the processor determines that the directly calculated physiological parameter is in doubt, and if the motion grade is the high motion grade, the processor determines that the directly calculated physiological parameter is ineffective.

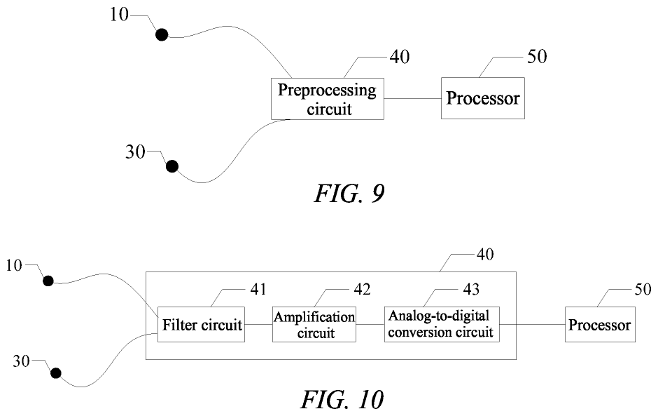

It can be seen from the above description of the processor 50 that the processor 50 is involved in determining the signal characteristic of the physiological signal and the signal characteristic of the motion signal. In an embodiment, the signal characteristic may include a time domain characteristic and/or a frequency domain characteristic of a signal. The time domain characteristic of the signal may include: at least one of a peak position, a peak amplitude, a peak slope, a peak width, peak effectiveness, a peak type, a peak-to-peak interval value, peak-to-peak interval effectiveness, and signal quality. There are many specific ways for the processor 50 to determine the signal characteristic of the signal. For example, the time domain signal is used as an example, where the processor 50 may perform peak searching on the signal, calculate a peak amplitude, a peak slope, and a peak width, and further calculate signal quality and other time domain characteristics such as peak effectiveness, a peak type, a peak-to-peak interval value, and interval effectiveness. For example, the frequency domain characteristic is used as an example, where the processor 50 may perform Fourier Transform on the signal to obtain a frequency characteristic of the signal, and then process a high-frequency signal and a low-frequency signal to obtain a required spectrum characteristic. In order to reduce hardware unlocking and costs, in an embodiment, referring to FIG. 8, the processor 50 may include a signal characteristic extraction unit 51 configured to: extract an input signal and output a signal characteristic of the input signal, extract a physiological signal and output a signal characteristic of the physiological signal when the input signal is the physiological signal, and extract a motion signal and output a signal characteristic of the motion signal when the input signal is the motion signal. Referring to FIG. 9, in an embodiment, in order to better obtain the signal characteristic of the signal, the monitoring device of the disclosure may further include a preprocessing circuit 40 configured to first preprocess the physiological signal and the motion signal in parallel before the processor determines the signal characteristic of the physiological signal from the physiological signal and determines the signal characteristic of the motion signal from the motion signal. For example, referring to FIG. 10, the preprocessing circuit 40 may include at least one of a filter circuit 41, an amplification circuit 42, and an analog-to-digital conversion circuit 43. The filter circuit 41 is configured to filter an input signal, the amplification circuit 42 is configured to amplify the input signal, and the analog-to-digital conversion circuit 43 is configured to perform analog-to-digital conversion on the input signal. The filter circuit 41 may be used as a front stage of the preprocessing circuit 40, the amplification circuit 42 may be used as an intermediate stage of the preprocessing circuit 40, and the analog-to-digital conversion circuit 43 may be used as a rear stage of the preprocessing circuit. For example, the preprocessing circuit 40 first filters the signal by means of the filter circuit 41 to remove noise, then amplifies the filtered signal by means of the amplification circuit 42 to enlarge a signal amplitude, and finally performs analog-to-digital conversion on the amplified signal by means of the analog-to-digital conversion circuit 43 to convert the signal from an analog signal to a digital signal.

It should be noted that for some structures and components of the monitoring device disclosed in FIG. 7 to FIG. 10, reference may also be made to the monitors disclosed in FIG. 1 and FIG. 2. For example, the processor 50 in the monitoring device in FIG. 7 to FIG. 10 may be implemented by the main processor 115 in FIG. 1 and FIG. 2. The physiological sensor 10 may refer to a sensor accessory 111. Moreover, the monitoring device disclosed in FIG. 7 to FIG. 10 may further include other components disclosed in FIG. 1 and FIG. 2, for example, the isolated power supply and communication interface 114 and the external communication and power interface 116. Details are not described herein again. In addition, the monitoring device disclosed in FIG. 7 to FIG. 10 may also be all or part of the portable monitoring apparatus 213 shown in FIG. 3. For example, some of the steps in FIG. 4 to FIG. 6 may be performed in the portable monitoring apparatus 213, and some may be performed in the bedside monitor 212. Alternatively, all the steps in FIG. 4 to FIG. 6 are performed in the portable monitoring apparatus 213, and the processed data is displayed, output, and stored by means of the central station 211 or the bedside monitor 212.

The monitoring device disclosed in the disclosure may be a mobile monitoring device, so that the requirements of mobile monitoring can be satisfied without limiting an activity space of a patient. In addition, for a conventional monitoring device, interference may be caused by electrode pulling due to activities such as walking, getting in and out of bed, rubbing clothes by the patient. This may seriously interfere with a waveform signal, and may also affect accuracy of physiological parameter measurement, thereby further affecting the determination of the patient's condition by a doctor and affecting rehabilitation of the patient. In the disclosure, a physiological signal and a motion signal of a patient are acquired synchronously, a signal characteristic of the physiological signal is determined from the physiological signal, and whether the physiological signal is reliable is determined according to the signal characteristic of the physiological signal. If it is determined that the physiological signal is unreliable, a signal characteristic of the motion signal is determined from the motion signal, and a motion state during each heartbeat period of the monitored object is determined according to the signal characteristic of the motion signal. According to the motion state during the heartbeat period, the effectiveness of the physiological signal during the heartbeat period is determined, or the signal characteristic of the physiological signal during the heartbeat period is corrected when a physiological parameter is calculated, thereby satisfying the measurement during mobile monitoring and ensuring measurement performance.

The above description is the method for optimizing physiological parameter using a motion sensor and the monitoring device disclosed in the disclosure, and a practical example is given below for description.

For example, the physiological sensor 10 is an electrocardiogram sensor attached to a monitored object, and an acquired physiological signal is an ECG signal. Therefore, the ECG signal of the monitored object is acquired by means of the electrocardiogram sensor attached to the monitored object, and a motion signal of the monitored object is synchronously acquired by means of a motion sensor attached to the monitored object.

A signal characteristic of the ECG signal is determined from the ECG signal, where the signal characteristic of the ECG signal may include: an amplitude, slope, width, frequency and the like of a QRS complex, effectiveness and type of the QRS complex, a peak-to-peak interval value, interval value effectiveness, signal quality, and the like.

Whether the ECG signal is reliable is determined according to the signal characteristic of the ECG signal, for example, statistics collection is performed on signal characteristics of the ECG signal, voting and scoring are performed based on different weights of these signal characteristics, a reliability score of the ECG signal is acquired, and whether the ECG signal is reliable is determined based on the acquired reliability score.

When it is determined that the ECG signal is reliable, an ECG parameter such as a heart rate may be directly calculated based on the signal characteristic of the ECG signal.

When it is determined that the ECG signal is unreliable, optimization may be performed according to the motion signal.

Specifically, a signal characteristic of the motion signal is determined from the motion signal, and a motion state during each heartbeat period of the monitored object is determined according to the signal characteristic of the motion signal. For example, the motion state during each heartbeat period of the monitored object is graded according to the signal characteristic of the motion signal, to obtain a motion grade during each heartbeat period of the monitored object. For example, the motion grade may include three grades: a low motion grade for indicating that the monitored object has no motion or slight motion, a high motion grade for indicating that the monitored object has intense motion, and a medium motion grade between the low motion grade and the high motion grade.

According to the motion state during the heartbeat period, the effectiveness of the ECG signal during the heartbeat period is determined, or the signal characteristic of the ECG signal during the heartbeat period is corrected when calculating the ECG parameter. Each heartbeat period corresponds to a QRS waveform. For example, when the motion grade is the low motion grade, no extra processing is performed on the ECG signal, and the signal characteristic of the ECG signal may be directly output; when the motion grade is the medium motion grade, a current QRS complex interval of the ECG signal may be corrected to be invalid, the signal quality of the ECG signal may be corrected to 3, and a current QRS complex type of the ECG signal may be determined again; and when the motion grade is the high motion grade, the QRS complex interval of the ECG signal may be directly corrected to be invalid, the signal quality of the ECG signal may be corrected to 4, and the QRS complex type of the electric signal may be corrected to be normal, to reduce the alarm of ventricular arrhythmia.

A person skilled in the art may understand that all or some of the functions of the various methods in the above implementations may be implemented by means of hardware or by means of a computer program. When all or some of the functions in the above implementations are implemented by means of a computer program, the program may be stored in a computer-readable storage medium, and the storage medium may include: a read-only memory, a random access memory, a magnetic disk, an optical disk, a hard disk, and the like, and the program is executed by a computer to implement the above functions. For example, the program is stored in a memory of an apparatus, and when the program in the memory is executed by means of a processor, all or some of the above functions can be implemented. In addition, when all or some of the functions in the above implementations are implemented by means of a computer program, the program may also be stored in a storage medium such as a server, another computer, a magnetic disk, an optical disk, a flash disk, or a mobile hard disk, may be saved in a memory of a local apparatus through downloading or copying, or version updating may be performed on a system of the local apparatus. When the program in the memory is executed by means of a processor, all or some of the functions in the above implementations can be implemented.

The disclosure is described by using specific examples above, which are merely for the purpose of facilitating understanding of the disclosure and are not intended to limit the disclosure. For a person of ordinary skill in the art, changes may be made to the above specific implementations according to the idea of the disclosure.

What is claimed is:

1. A method for optimizing physiological parameter using a motion sensor, comprising:
acquiring a physiological signal of a monitored object by a physiological sensor attached to the monitored object, and synchronously acquiring a motion signal of the monitored object by a motion sensor attached to the monitored object;
determining a signal characteristic of the physiological signal from the physiological signal, and determining whether the physiological signal is reliable according to the signal characteristic of the physiological signal;
when determining the physiological signal is unreliable, determining a signal characteristic of the motion signal from the motion signal, and determining a motion state during each heartbeat period of the monitored object during a time period in which the motion signal is acquired according to the signal characteristic of the motion signal; and
according to the motion state during the heartbeat period, determining an effectiveness of the physiological signal during the heartbeat period, or correcting the signal characteristic of the physiological signal during the heartbeat period when calculating a physiological parameter of the monitored object, or directly correcting the physiological parameter of the monitored object.

2. The method of claim 1, further comprising providing a signal characteristic extraction unit that is configured to extract and output a signal characteristic of an input signal inputted to the signal characteristic extraction unit; wherein the signal characteristic extraction unit is configured to extract and output the signal characteristic of the physiological signal when the input signal is the physiological signal, and extract and output the signal characteristic of the motion signal when the input signal is the motion signal.

3. The method of claim 1, wherein before the signal characteristic of the physiological signal is determined from the physiological signal and the signal characteristic of the motion signal is determined from the motion signal, the method further comprises: preprocessing the physiological signal and the motion signal.

4. The method of claim 3, wherein preprocessing the physiological signal and the motion signal comprises: performing at least one of filtering, amplification, and analog-to-digital conversion on the physiological signal and the motion signal.

5. The method of claim 1, wherein determining whether the physiological signal is reliable according to the signal characteristic of the physiological signal comprises: assigning a weight to each signal characteristic of the physiological signal, acquiring a reliability score of the physiological signal through voting and scoring, and determining whether the physiological signal is reliable based on the reliability score.

6. The method of claim 1, wherein determining a motion state during each heartbeat period of the monitored object during a time period in which the motion signal is acquired according to the signal characteristic of the motion signal comprises: grading the motion state during each heartbeat period of the monitored object during the time period in which the motion signal is acquired according to the signal characteristic of the motion signal, to obtain a motion grade during each heartbeat period of the monitored object.

7. The method of claim 6, wherein grading the motion state during each heartbeat period of the monitored object during the time period in which the motion signal is acquired according to the signal characteristic of the motion signal, to obtain a motion grade during each heartbeat period of the monitored object comprises: calculating a mean value or a variance of the signal characteristics of the motion signal, and grading the motion state during each heartbeat period of the monitored object during a time period in which the motion signal is acquired through threshold segmentation, to obtain the motion grade during each heartbeat period of the monitored object.

8. The method of claim 6, wherein the motion grade comprises: a low motion grade for indicating that the monitored object has no motion or slight motion, a high motion grade for indicating that the monitored object has intense motion, and a medium motion grade between the low motion grade and the high motion grade.

9. The method of claim 6, wherein determining an effectiveness of the physiological signal during the heartbeat period, or correcting the signal characteristic of the physiological signal during the heartbeat period when calculating a physiological parameter of the monitored object, or directly correcting the physiological parameter of the monitored object, according to the motion state during the heartbeat period comprises: determining an effectiveness of the physiological signal according to the motion grade during the heartbeat period, or correcting the signal characteristic of the physiological signal during the heartbeat period according to the motion grade during the heartbeat period when calculating the physiological parameter of the monitored object, or directly correcting the physiological parameter according to the motion grade during the heartbeat period of the monitored object.

10. The method of claim 9, wherein the signal characteristic comprises signal quality; and correcting the signal characteristic of the physiological signal during the heartbeat period according to the motion grade during the heartbeat period when calculating the physiological parameter of the monitored object comprises: correcting the signal quality of the physiological signal during the heartbeat period according to the motion grade during the heartbeat period when calculating the physiological parameter of the monitored object.

11. The method of claim 9, determining an effectiveness of the physiological signal according to the motion grade during the heartbeat period comprises: when the motion grade during the heartbeat period is a low motion grade for indicating that the monitored object has no motion or slight motion, determining that the physiological signal during the heartbeat period is effective; when the motion grade during the heartbeat period is a high motion grade for indicating that the monitored object has intense motion, determining that the physiological signal during the heartbeat period is ineffective; and when the motion grade during the heartbeat period is a medium motion grade between the low motion grade and the high motion grade, reconfirming the effectiveness of the physiological signal during the heartbeat period;
or, correcting the signal characteristic of the physiological signal during the heartbeat period according to the motion grade during the heartbeat period when calculating the physiological parameter of the monitored object comprises: when the motion grade during the heartbeat period is a low motion grade for indicating that the monitored object has no motion or slight motion, the signal characteristic of the physiological signal is not modified when calculating the physiological parameter of the monitored object; and when the motion grade during the heartbeat period is a high motion grade for indicating that the monitored object has intense motion or a medium motion grade between the low motion grade and the high motion grade, correcting the signal characteristic of the physiological signal when calculating the physiological parameter of the monitored object.

12. The method of claim 1, wherein the signal characteristic comprises a time domain characteristic or a frequency domain characteristic of a signal; and the time domain characteristic of the signal comprises: at least one of a peak position, a peak amplitude, a peak slope, a peak width, peak effectiveness, a peak type, a peak-to-peak interval value, peak-to-peak interval effectiveness, and signal quality.

13. The method of claim 12, wherein correcting the signal characteristic of the physiological signal during the heartbeat period when calculating a physiological parameter of the monitored object comprises: correcting at least one weight of a time domain characteristic or a frequency domain characteristic of the physiological signal during the heartbeat period when calculating the physiological parameter of the monitored object.

14. The method of claim 1, further comprising: when determining the physiological signal is reliable, directly calculating the physiological parameter based on the signal characteristic of the physiological signal; or,
when determining the physiological signal is unreliable, the method further comprises: directly calculating the physiological parameter of the monitored object based on the signal characteristic of the physiological signal determined from the physiological signal; and determining whether the calculated physiological parameter during the heartbeat period is effective according to the effectiveness of the physiological signal during the heartbeat period.

15. A monitoring device, comprising:
a physiological sensor configured to be attached to a monitored object to acquire a physiological signal of the monitored object;
a motion sensor configured to be attached to the monitored object to acquire a motion signal of the monitored object; and
a processor configured to: determine a signal characteristic of the physiological signal from the physiological signal, and determine , whether the physiological signal is reliable according to the signal characteristic of the physiological signal; when the physiological signal is unreliable, determine a signal characteristic of the motion signal from the motion signal, and determine a motion state during each heartbeat period of the monitored object during a time period in which the motion signal is acquired according to the signal characteristic of the motion signal; and according to the motion state during the heartbeat period, determine an effectiveness of the physiological signal during the heartbeat period, or correct the signal characteristic of the physiological signal during the heartbeat period when calculating a physiological parameter of the monitored object, or directly correct the physiological parameter of the monitored object.

16. The monitoring device of claim 15, wherein the processor comprises a signal characteristic extraction unit configured to: extract an input signal and output a signal characteristic of the input signal, extract the physiological signal and output a signal characteristic of the physiological signal when the input signal is the physiological signal, and extract the motion signal and output the signal characteristic of the motion signal when the input signal is the motion signal.

17. The monitoring device of claim 16, further comprising a preprocessing circuit configured to preprocess the physiological signal and the motion signal before the processor determines the signal characteristic of the physiological signal from the physiological signal and determines the signal characteristic of the motion signal from the motion signal.

18. The monitoring device of claim 17, wherein the preprocessing circuit comprises at least one of the following:
a filter circuit configured to filter the input signal;
an amplification circuit configured to amplify the input signal; and
an analog-to-digital conversion circuit configured to perform analog-to-digital conversion on athe input signal.

19. The monitoring device of claim 15, further comprising a housing, wherein the processor is arranged in the housing and is connected to the physiological sensor and the motion sensor in a wired or wireless manner.

20. The monitoring device of claim 15, wherein the motion sensor comprises at least one of an acceleration sensor, an angular velocity sensor, or a gravity sensor.
